(19) United States
(12) Patent Application Publication
(10) Pub. No.: US 2026/0249016 A1
(43) Pub. Date: Aug. 27, 2026

(54) DEVICES THAT FACILITATE INSERTION OF CENTRAL VENOUS CATHETERS

(71) Applicant: The University of North Carolina at Chapel Hill, Chapel Hill, NC (US)

(72) Inventors: Ben SMITH, Durham, NC (US); Grayson HINNANT, Cary, NC (US); McKenna WELCH, Raleigh, NC (US); Alex KONZMANN, Cranston, RI (US); Callum FUNK, Baltimore, MD (US); Emily WARREN, Cary, NC (US)

(21) Appl. No.: 19/548,562

(22) Filed: Feb. 24, 2026

Related U.S. Application Data
(60) Provisional application No. 63/763,533, filed on Feb. 26, 2025.

(51) Int. Cl. A61M 5/315; A61M 5/31

(52) U.S. Cl. CPC A61M 5/31578; A61M 2005/3131; A61M 2005/3139; A61M 2205/586; A61M 2210/12

(57) ABSTRACT

A syringe includes a barrel, a plunger slidably mounted within the barrel chamber, and an elongate rod movably secured to the barrel. The rod is attached to the plunger and is configured to be engaged by a finger of a user holding the syringe for bidirectional movement of the plunger. A device includes a frame configured to be attached to a syringe barrel, and an elongate rod movably secured to the frame. The rod is configured to be engaged by a finger of a user holding the syringe for bidirectional movement of the plunger. A device includes an elongate rod having a proximal end configured to be secured to a thumb press of a plunger within a barrel of a syringe, and a distal end configured to be engaged by a finger of a user holding the syringe for bidirectional movement of the plunger within the barrel.

### RELATED APPLICATION

This application claims the benefit of and priority to U.S. Provisional Patent Application No. 63/763,533 filed February 26, 2025, the disclosure of which is incorporated herein by reference as if set forth in its entirety.

### FIELD OF THE INVENTION

The present invention relates generally to medical catheters and, more particularly, to devices for inserting a medical catheter in a patient.

### BACKGROUND OF THE INVENTION

A central venous catheter (CVC) provides long term vascular access for diagnostic or therapeutic treatments such as blood sampling, central venous pressure readings, administration of fluid or medications, total parenteral nutrition, and blood transfusions. Additionally, CVCs may be used for pulmonary artery catheter introduction and transvenous pacing wire placement. A patient may develop a need for a CVC through a pre-existing disease state, a developing disease state, or a trauma incident. Once it has been determined necessary for a patient to receive a CVC, the CVC is inserted into the patient by a trained health care provider (e.g., a physician, physician assistant, a nurse practitioner, etc.) and appropriate materials are introduced through the catheter port(s).

FIG. 1 illustrates different insertion cites for CVCs in the human body. A CVC is typically inserted into the internal jugular vein, the subclavian vein, axillary vein, or the femoral vein. The location of the CVC placement will depend on patient state or injury and physician preference. The left subclavian vein and the right internal jugular vein provide the most direct paths to the heart, but all three of these veins will carry any injected materials directly to the heart to be pumped out to the rest of the body.

Referring to FIGS. 2A and 2B, when a CVC is placed, an introducer needle is first inserted into the skin at the chosen site to find the appropriate vein. Once the location has been confirmed through ultrasound-guided imaging and/or aspirating venous blood through an attached syringe, a guidewire is advanced through the needle to the vein. A catheter is finally threaded over the wire into the vein. After the catheter has been successfully placed, fluids, medication, and any blood transfusions necessary for patient treatment can be administered through the CVC port and directly into the appropriate vein.

Unfortunately, one complication from CVC placement that may occur is central line associated blood stream infection (CLABSI), which can form at the catheter tip and can grow and travel through the bloodstream to other organs of the body. In addition, various mechanical complications may occur when there are errors in placement of a CVC, such as arterial puncture and lung puncture. When an introducer needle of a syringe punctures an artery instead of the appropriate vein, blood may pool in the body cavity, which may cause a hemorrhage or a hematoma to form. If an introducer needle of a syringe punctures a lung, air may be allowed into the pleural cavity, pushing on the outside of the lung and causing the lung to collapse.

Patients receiving CVCs are at an elevated risk for mechanical complications in environments where ultrasound guidance is unavailable. For example, ultrasound-guided insertion has a complication rate of only 4.6%, while landmark (i.e., no ultrasound) has a complication rate of 16.9%. In developing countries, austere medical environments, and emergency situations, ultrasound may be unavailable during CVC placement. Patients treated in hospitals that have limited access to ultrasound machines are primarily affected by complications during blind CVC insertion. As such, there is a need for a way to more accurately place CVCs in patients requiring central venous access, particularly where ultrasound guidance is unavailable, in order to reduce the incidence of mechanical complications.

### SUMMARY OF THE INVENTION

It should be appreciated that this Summary is provided to introduce a selection of concepts in a simplified form, the concepts being further described below in the Detailed Description. This Summary is not intended to identify key features or essential features of this disclosure, nor is it intended to limit the scope of the invention.

According to some embodiments of the present invention, a syringe includes a barrel defining a longitudinally extending chamber and having open proximal and distal ends. The barrel distal end includes a wall with a tip extending distally therefrom that serves as a needle hub. The tip includes a passageway therethrough that is in fluid communication with the chamber. A plunger is slidably mounted within the chamber, and a stopper is secured to a distal end of the plunger to form a fluid-tight seal with a surface of the chamber. Fluid contained within the chamber is expelled through the open distal end of the barrel upon movement of the stopper toward the distal end of the barrel, and fluid may be drawn through the open distal end of the barrel into the chamber upon movement of the stopper toward the proximal end of the barrel. An elongate rod is movably secured to an outer portion of the barrel. The rod includes a proximal end attached to the plunger, a distal end, and a finger platform adjacent the rod distal end that is configured to be engaged by a finger of a user holding the syringe. Bidirectional movement of the rod via the finger platform causes corresponding bidirectional movement of the plunger within the barrel.

In some embodiments, the barrel has a longitudinally extending guide that is integral with an outer surface thereof, and the rod is slidably retained within the guide.

In some embodiments, the finger platform has an ergonomic configuration that facilitates engagement by the finger of the user. For example, the finger platform may include a rounded periphery, a flat distal surface, and a curved proximal surface. In some embodiments, the distal surface of the finger platform is flush with the rod distal end.

In some embodiments, the plunger proximal end includes a thumb press, and the rod proximal end is attached to the thumb press.

According to other embodiments of the present invention, a device for operating a syringe includes a frame configured to be attached to a barrel of the syringe, and an elongate rod movably secured to the frame. The rod includes a proximal end, a distal end, and a finger platform configured to be engaged by a finger of a user holding the syringe. The rod proximal end is configured to engage a portion of a plunger extending outward from the barrel proximal end. In some embodiments, the frame includes a longitudinally extending guide on an outer surface thereof, and the rod is slidably retained within the guide. Bidirectional movement of the rod via the finger platform causes corresponding bidirectional movement of the plunger within the barrel of the syringe.

In some embodiments, the frame includes a first clip configured to be removably secured to a distal end portion of the syringe barrel, and a second clip configured to be removably secured to a proximal end portion of the syringe barrel.

In some embodiments, the proximal end of the syringe barrel includes a peripheral flange and the frame includes an adapter configured to surround a portion of the barrel and engage the peripheral flange. In some embodiments, the adapter includes a pair of spaced-apart clips that are configured to receive the portion of the peripheral flange therebetween.

In some embodiments, the plunger includes a thumb press, and the rod proximal end is configured to engage the thumb press. For example, the rod proximal end may include a member with a slot, and a portion of the thumb press is received within the slot.

In some embodiments, the finger platform has an ergonomic configuration that facilitates engagement by a finger of the user. For example, the finger platform may include a rounded periphery, a flat distal surface, and a curved proximal surface. In some embodiments, the distal surface of the finger platform is flush with the rod distal end.

According to some embodiments of the present invention, a device for operating a syringe includes an elongate rod having a proximal end and an opposite distal end. The the proximal end of the rod is removably secured to a thumb press of a plunger that is movably secured within a barrel of the syringe, and the distal end of the rod includes a finger platform that is configured to be engaged by a finger of a user holding the syringe. Bidirectional movement of the rod via the finger platform causes corresponding bidirectional movement of the plunger within the barrel of the syringe.

In some embodiments, the rod proximal end includes a member with a slot, and a portion of the thumb press is received within the slot.

In some embodiments, the rod distal end includes a member configured to slide along an outer surface of the barrel of the syringe during movement of the rod.

In some embodiments, the rod distal end includes an arcuate member that at least partially surrounds the barrel of the syringe and is configured to slide along an outer surface of the barrel of the syringe during movement of the rod.

Embodiments of the present invention allow for one-handed use of a syringe during insertion of a CVC into a patient, and provide increased hand stability and control of needle movement during the first two steps of the CVC insertion procedure: confirming venous access by pulling negative pressure, and advancing the guide-wire into the central vein. This increased stability and control may reduce the potential for mechanical complications and may reduce the need for emergency procedures to combat internal puncture wounds.

A one way valve plunger may be used with embodiments of the present invention to create a closed system, essentially removing the risk of air embolism. A guide-wire attachment may be used with embodiments of the present invention to allow for easy access of the guide-wire during the multi-step Seldinger technique process, thereby decreasing the procedure time.

Embodiments of the present invention may be used for any procedure using the Seldinger technique (such as pigtail chest tube placement). Embodiments of the present invention could also be used for other procedures requiring aspiration such as arthrocentesis, thoracentesis, or paracentesis. Embodiments of the present invention are not limited for use only with CVC insertion.

It is noted that aspects of the invention described with respect to one embodiment may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present invention. The drawings and description together serve to fully explain embodiments of the present invention.

FIG. 1 is an illustration of the anatomy of the human body showing different insertion sites for a CVC.

FIGS. 2A-2B are illustrations of the introducer needle stage of a CVC insertion procedure for the internal jugular vein with ultrasound guided-image assistance.

FIGS. 3-5 are top perspective views of a syringe for assisting insertion of a CVC into a patient, according to some embodiments of the present invention.

FIG. 6 is a front end view of the syringe of FIGS. 3-5.

FIG. 7A is a top plan view of the syringe of FIGS. 3-5.

FIG. 7B is a side view of the syringe of FIGS. 3-5.

FIG. 8 is a perspective view of the syringe of FIGS. 3-5 showing the plunger moved toward the proximal end of the barrel chamber.

FIGS. 9-10 are top perspective views of a device for operating a syringe for assisting insertion of a CVC into a patient, according to some embodiments of the present invention.

FIG. 11A is a side view of the device of FIGS. 9-10.

FIG. 11B is a top view of the device of FIGS. 9-10.

FIG. 12 is a bottom perspective view of the device of FIGS. 9-10.

FIGS. 13-14 illustrate the device of FIGS. 9-10 attached to a syringe and being held by a user in a first operative position.

FIGS. 15-16 illustrate the device of FIGS. 9-10 attached to a syringe and being held by a user in a second operative position.

FIG. 17 is a top perspective view of a device for operating a syringe for assisting insertion of a CVC into a patient, according to some embodiments of the present invention.

FIG. 18 is a bottom perspective view of the device of FIG. 17.

FIG. 19 is a top perspective view of a device for operating a syringe for assisting insertion of a CVC into a patient, according to some embodiments of the present invention.

FIG. 20 is a bottom perspective view of the device of FIG. 19.

FIG. 21 is a top perspective view of a device for operating a syringe for assisting insertion of a CVC into a patient, according to some embodiments of the present invention.

FIGS. 22A-22B illustrate the device of FIG. 21 operably associated with the barrel of a syringe.

FIG. 23 is a top perspective view of a device for operating a syringe for assisting insertion of a CVC into a patient, according to some embodiments of the present invention.

FIG. 24 is a bottom perspective view of the device of FIG. 23.

FIGS. 25A-25B illustrate the device of FIGS. 23-24 operably associated with the barrel of a syringe.

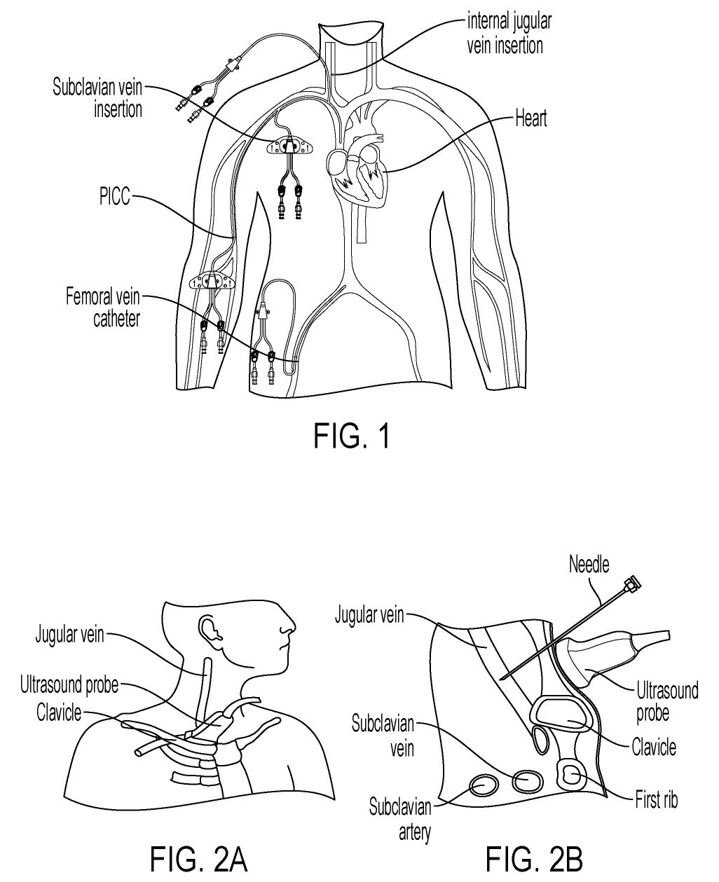

### DETAILED DESCRIPTION OF THE INVENTION

The present invention will now be described more fully hereinafter with reference to the accompanying figures, in which embodiments of the present invention are shown. The present invention may, however, be embodied in many different forms and should not be construed as limited to the embodiments set forth herein.

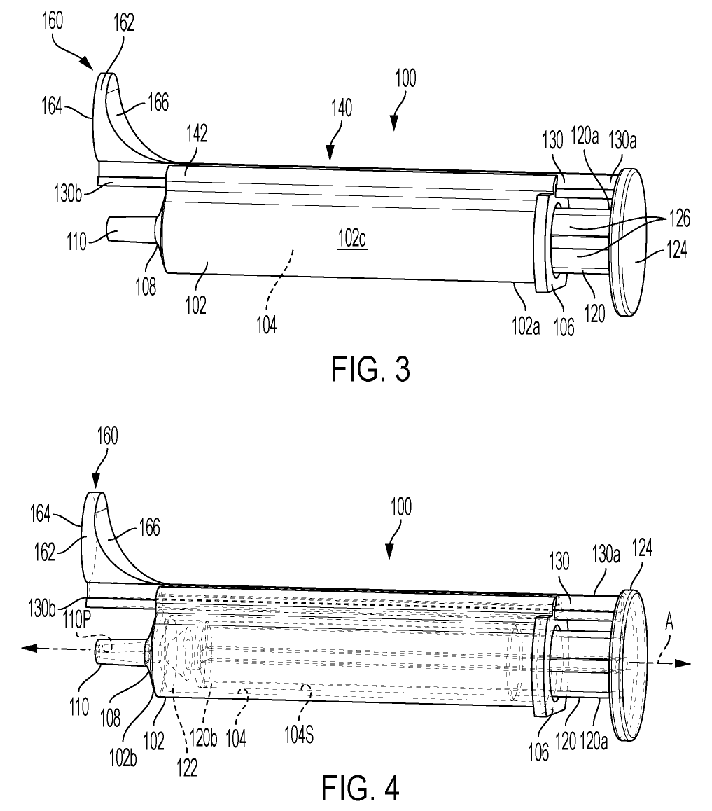

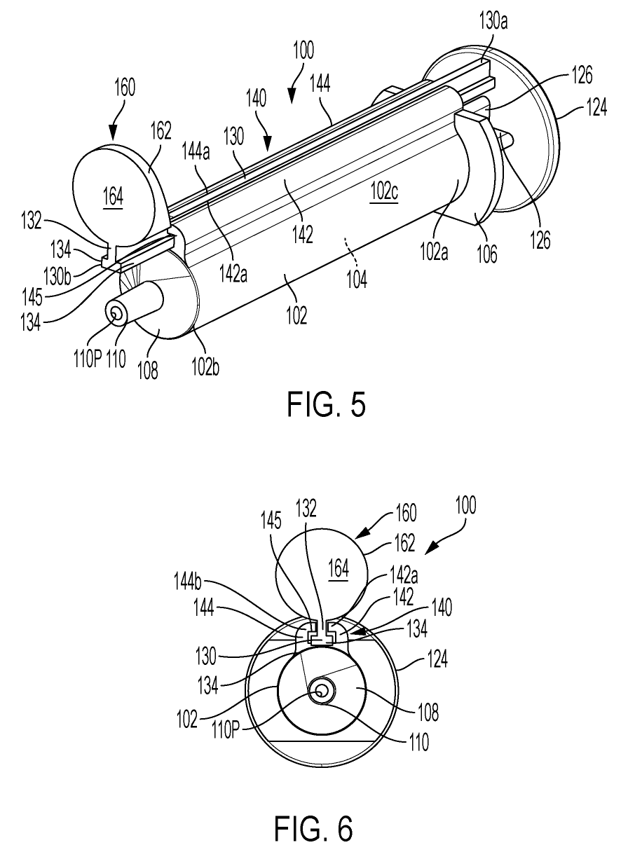

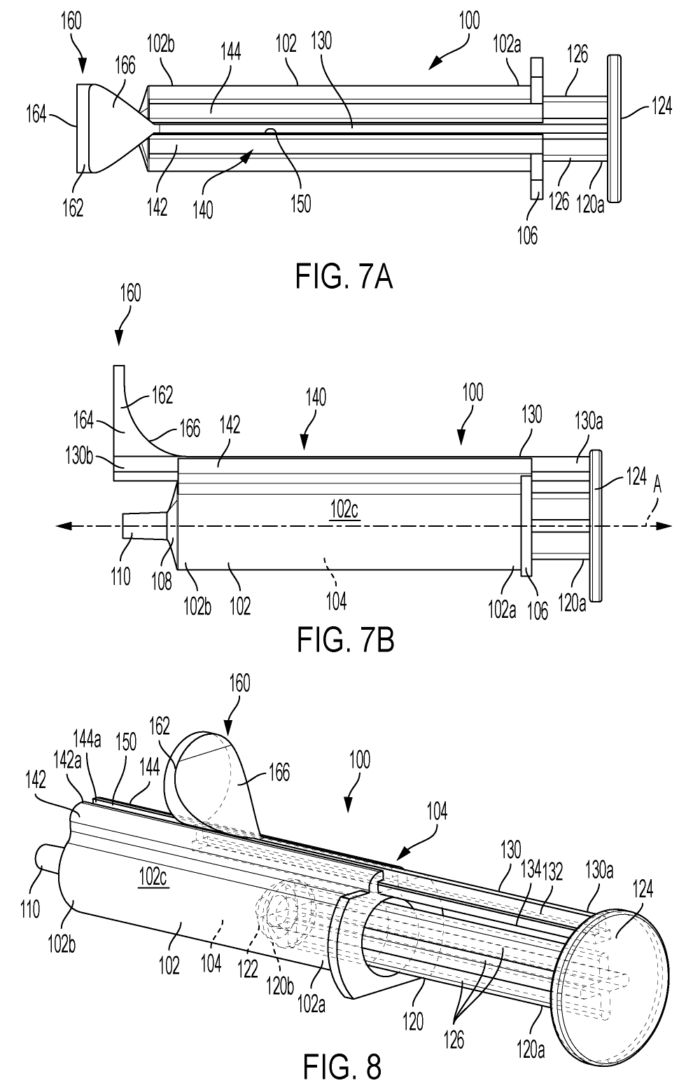

Referring to FIGS. 3-8, a syringe 100 for assisting insertion of a CVC into a patient, according to some embodiments of the present invention, is illustrated. As used herein, the term “distal end” refers to the end of the syringe 100 furthest from a user holding the syringe 100. The term “proximal end” refers to the end of the syringe 100 closest to the holder of the syringe 100. The illustrated syringe 100 includes a generally cylindrical barrel 102 that is hollow and that defines a longitudinally extending chamber 104. The barrel 102 includes an open proximal end 102a and an open distal end 102b. A peripheral flange 106 is integrally formed on the outer surface of the barrel 102 at the proximal end 102a and extends radially outward from the barrel 102, as illustrated. The barrel distal end 102b includes a wall 108 with a tip 110 extending distally therefrom that serves as a needle hub. The tip 110 includes a passageway 110p formed therethrough that is in fluid communication with the chamber 104.

A plunger 120 is slidably mounted within the chamber 104. The plunger 120 has an elongate configuration with a distal end 120b and an opposite proximal end 120a. A stopper 122 is secured to the distal end 120b of the plunger 120 to form a fluid-tight seal with an inner surface 104s of the chamber 104. The stopper 122 may be made of a polymeric material, such as rubber for example, and is friction fit to the inner surface 104s of the chamber 104. As such, fluid contained within the chamber 104 can be expelled through the open distal end 102b of the barrel (and through the tip 110) upon movement of the stopper 122 toward the distal end 102b of the barrel 102, and fluid may be drawn through the tip 110 and the open distal end 102b of the barrel 102 upon movement of the stopper 122 toward the proximal end 102a of the barrel 102.

The proximal end 120a of the plunger 120 includes a thumb press 124 that can be pressed by a user to move the plunger 120 toward the distal end 102b of the barrel 102, and can be gripped and pulled by a user to move the plunger 120 toward the proximal end 102a of the barrel 102. In the illustrated embodiment, the plunger includes four elongate ribs 126 that extend radially from a longitudinal center axis A of the plunger 120. However, other embodiments of the plunger may have different numbers of ribs. In addition, the plunger 120 may have various other shapes and configurations. Embodiments of the present invention are not limited to the illustrated shape of the plunger 120.

The syringe barrel 102 and plunger 120 may be formed from various materials. In some embodiments, the barrel 102 and/or plunger 120 may be formed from one or more polymeric materials, such a polypropylene, polyethylene, etc. In other embodiments, the barrel 102 and/or plunger 120 may be formed from non-polymeric materials, such as glass or stainless steel, etc.

An elongate rod 130 is movably secured to an outer portion of the barrel 102, as illustrated. The rod 130 includes a proximal end 130a that is attached to a portion of the plunger 120 extending outward from the proximal end 102a of the barrel 102, and an opposite distal end 130b. In the illustrated embodiment, the proximal end 130a of the rod 130 is attached to the thumb press 124 of the plunger 120. A finger platform 160 is attached to the rod 130 near the rod distal end 130b and is configured to be engaged by a finger of a user holding the syringe 100. Bidirectional movement of the rod 130 via the finger platform 160 causes corresponding bidirectional movement of the plunger 120 within the barrel chamber 104. In other words, moving the finger platform 160 toward the proximal end 102a of the barrel 102 causes the plunger 120 to move toward the proximal end 102a of the barrel 102. Similarly, moving the finger platform 160 toward the distal end 102b of the barrel 102 causes the plunger 120 to move toward the distal end 102b of the barrel 102.

In the illustrated embodiment, the barrel 102 includes a longitudinally extending guide 140 that is attached to the outer surface 102c of the barrel 102, and the rod 130 is slidably retained within the guide 140. In some embodiments, the guide 140 may be integrally formed with the barrel 102 (i.e., the barrel 102 and guide 140 may be a unitary, monolithic structure). The illustrated guide 140 includes opposing, spaced apart elongate walls 142, 144 that are configured to define a channel 145 within which the rod 130 is retained and allowed to slide longitudinally relative to the barrel 102. The elongate end portions 142a, 144a of the walls 142, 144 are in face-to-face spaced apart relationship and form an elongate slot 150 therebetween, as illustrated. The rod 130 has a T-shaped cross section with an upper portion 132 and side portions 134 on either side of the upper portion 132. The side portions 134 of the rod 130 are retained within the channel 145 of the guide 140 and the upper portion 132 of the rod 130 is positioned in the slot 150 between the elongate end portions 142a, 144a of the walls 142, 144.

The finger platform 160 has an ergonomic configuration that facilitates engagement by a finger (e.g., the index finger) of the user. For example, in the illustrated embodiment, the finger platform 160 has a rounded periphery 162, a flat distal surface 164, and a curved proximal surface 166. In the illustrated embodiment, the distal surface 164 of the finger platform 160 is flush with the rod distal end 130b.

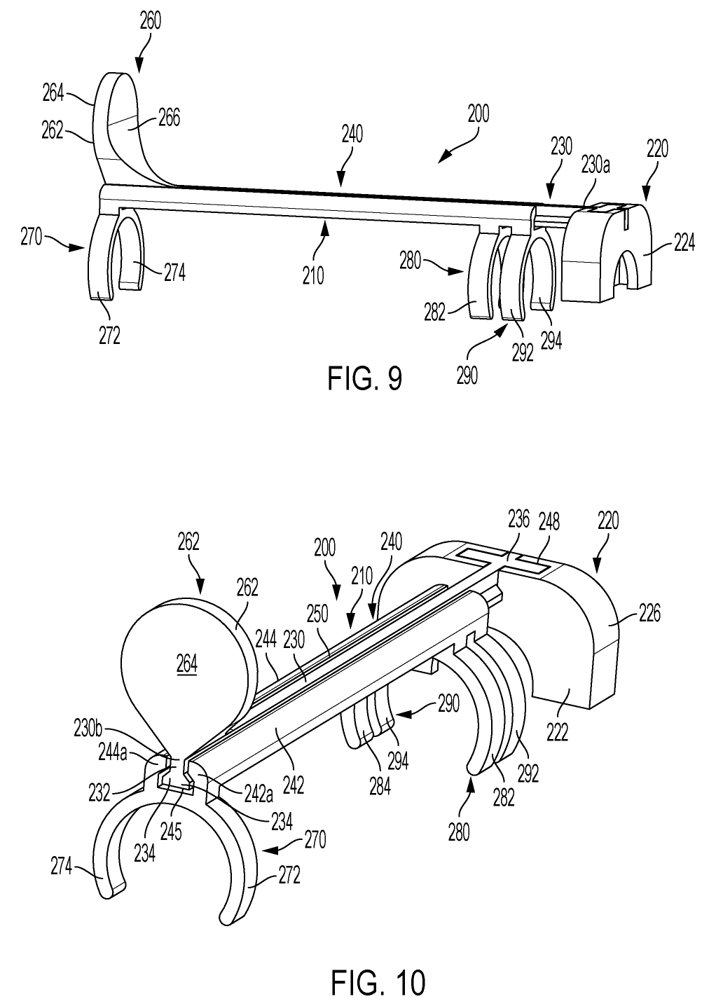

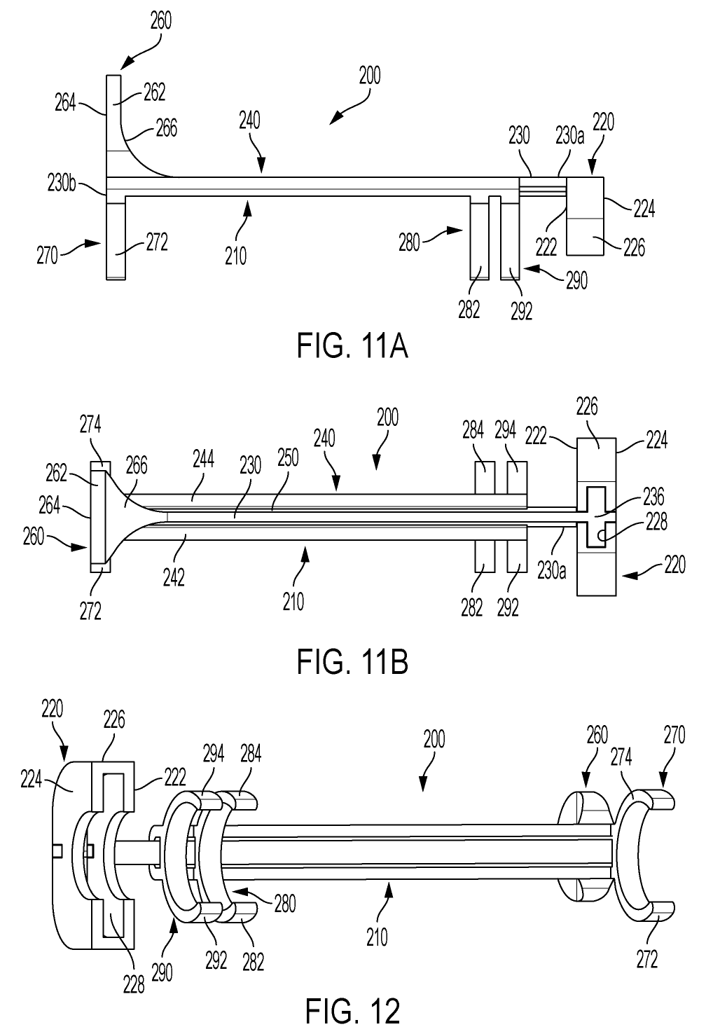

Referring to FIGS. 9-12, a device 200 for operating a syringe used in assisting insertion of a CVC into a patient, according to some embodiments of the present invention, is illustrated. The illustrated device 200 includes a frame 210 that is configured to be attached to a barrel of a syringe, and an elongate rod 230 movably secured to the frame 210. The rod 230 includes a proximal end 230a, a distal end 230b, and a finger platform 260 near the rod distal end 230b that is configured to be engaged by a finger of a user holding the syringe.

In the illustrated embodiment, the frame 210 has a longitudinally extending guide 240 and the rod 230 is slidably retained within the guide 240. The illustrated guide 240 includes opposing, spaced apart elongate walls 242, 244 that are configured to define a channel 245 within which the rod 230 can be retained and slide longitudinally relative to a barrel of a syringe. The elongate end portions 242a, 244a of the walls 242, 244 are in face-to-face spaced apart relationship to form an elongate slot 250, as illustrated. The rod 230 has a T-shaped cross section with an upper portion 232 and side portions 234 on either side of the upper portion 232. The side portions 234 of the rod 230 are retained within the channel 245 and the upper portion 232 of the rod 230 is positioned in the slot 250 between the elongate end portions 242a, 244a of the walls 242, 244.

The finger platform 260 is similar to the finger platform 160 described above, and has an ergonomic configuration that facilitates engagement by a finger (e.g., the index finger) of the user for operating the plunger of a syringe. In the illustrated embodiment, the finger platform 260 has a rounded periphery 262, a flat distal surface 264, and a curved proximal surface 266. In the illustrated embodiment, the distal surface 264 of the finger platform 260 is flush with the rod distal end 230b.

The illustrated frame 210 includes a first clip 270 that is configured to be removably secured to a distal end portion of a syringe barrel, and a pair of closely spaced-apart second and third clips 280, 290 that are configured to be removably secured to a proximal end portion of the syringe barrel. The illustrated clips 270, 280, 290 each have arcuate, flexible legs that are configured to straddle and grip the barrel of a syringe. For example, the first clip 270 includes arcuate, flexible first and second legs 272, 274 that are sized and configured to partially surround and snugly grip a portion of a syringe barrel. Similarly, the second and third clips 280, 290 include respective arcuate, flexible first and second legs 282, 284 and 292, 294 that are sized and configured to partially surround and snugly grip respective proximal end portions of a syringe barrel.

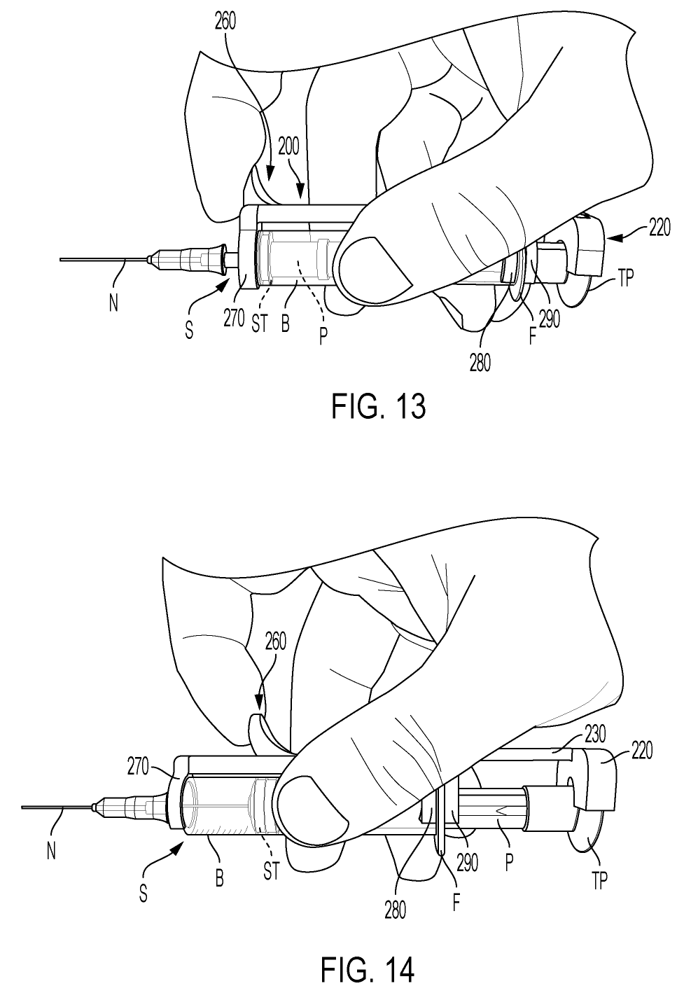

As illustrated in FIGS. 13-16, the proximal end of a syringe barrel B includes a peripheral flange F and the second and third and third clips 280, 290 are configured to receive the portion of the peripheral flange F therebetween. In the illustrated configuration, only the second clip 280 grips the barrel B of the syringe S and is configured to abut one side of the peripheral flange F. The third clip 290 is provided to engage an opposite side of the peripheral flange F. This engagement of the peripheral flange F by the second and third clips 280, 290 helps prevent longitudinal movement of the frame 210 relative to the barrel B of the syringe S during use of the syringe.

Referring back to FIGS. 9-12, the rod proximal end 230a is configured to engage a portion of a plunger of a syringe extending outward from the syringe barrel proximal end. Bidirectional movement of the rod 230 via the finger platform 260 causes corresponding bidirectional movement of the plunger within the barrel of the syringe. In other words, moving the finger platform 260 toward the proximal end of a barrel of a syringe to which the frame 210 is attached causes a plunger of the syringe to move toward the proximal end of the barrel of the syringe. Similarly, moving the finger platform 260 toward the distal end of the barrel of the syringe causes the plunger to move toward the distal end of the barrel.

In the illustrated embodiment, the rod proximal end 230a is attached to a member 220 that is configured to engage a thumb press at the end of a plunger of a syringe (e.g., thumb press TP illustrated in FIGS. 13-16). The illustrated member 220 includes a pair of spaced-apart walls 222, 224 with a connecting peripheral wall 226 that define a slot 228 within which a portion of a thumb press can be positioned. However, various ways of attaching the proximal end 230a of the rod to a thumb press or another portion of a plunger of a syringe may be utilized.

In the illustrated embodiment, the proximal end 230a of the rod 230 has a T-shaped portion 236 that is received and secured in a correspondingly-shaped portion 248 of the member 220. In some embodiments, the member 220 may be removably secured to the proximal end 230a of the rod 230 (i.e., the T-shaped portion 236 of the rod 230 may be removably secured to the correspondingly-shaped portion 248 of the member 220, for example, via a snap fit, etc.) This allows for members 220 of different shapes and configurations to be utilized so as to accommodate different thumb presses of various syringes. This allows for the device 200 to be utilized with a variety of syringes and not be limited to use with a particular type of syringe. However, in other embodiments, the rod 230 and member 220 may be formed as an integral unit.

The device 200 may be formed from various materials. In some embodiments, the device 200 may be formed from polymeric materials, such a polypropylene, polyethylene, etc. However, the device 200 may be formed from non-polymeric materials, such as stainless steel, etc.

FIGS. 13 and 14 illustrate the device 200 of FIGS. 9-12 attached to a syringe S, and the syringe S and device 200 being held by a user in a first operative position wherein the user’s hand H is positioned above the syringe S and device 200. The syringe S includes a barrel B, a plunger P with a stopper ST movably mounted within the barrel B, and a needle N secured to the distal end of the barrel B. The first clip 270 of the device 200 surrounds a portion of the distal end portion of the barrel B, the second clip 280 surrounds a portion of the proximal end of the barrel B, and the third clip 290 is on an opposite side of the barrel flange F from the second clip 280. The member 220 at the proximal end of the rod 230 is coupled to a portion of the thumb press TP at the proximal end of the plunger P.

In FIG. 13, the plunger stopper ST is positioned at the distal end of the barrel B of the syringe S, and the user’s index finger is positioned on the finger platform 260. In FIG. 14, the user has moved the finger platform 260 toward the proximal end of the syringe so as to move the plunger stopper ST toward the proximal end of the barrel B.

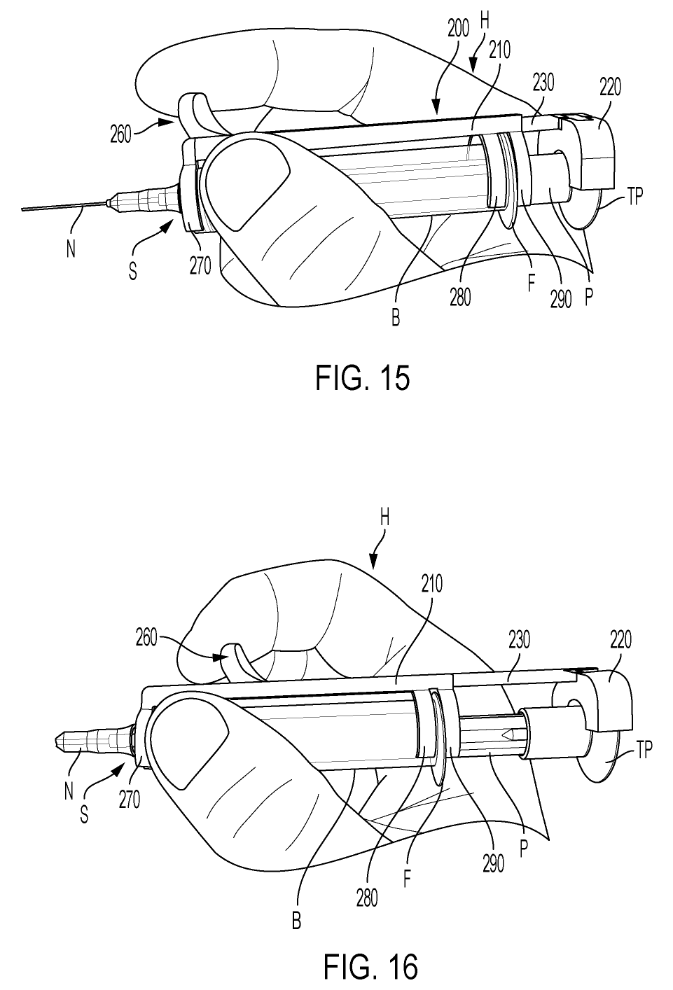

FIGS. 15 and 16 illustrate the device of FIGS. 9-10 attached to a syringe S and being held by a user in a second operative position wherein the user’s hand H is positioned below

the syringe S and device 200. In FIG. 15, the plunger stopper ST is positioned at the distal end of the barrel B of the syringe S, and the user’s index finger is positioned on the finger platform 260. In FIG. 16, the user has moved the finger platform 260 toward the proximal end of the syringe so as to move the plunger stopper ST toward the proximal end of the barrel B.

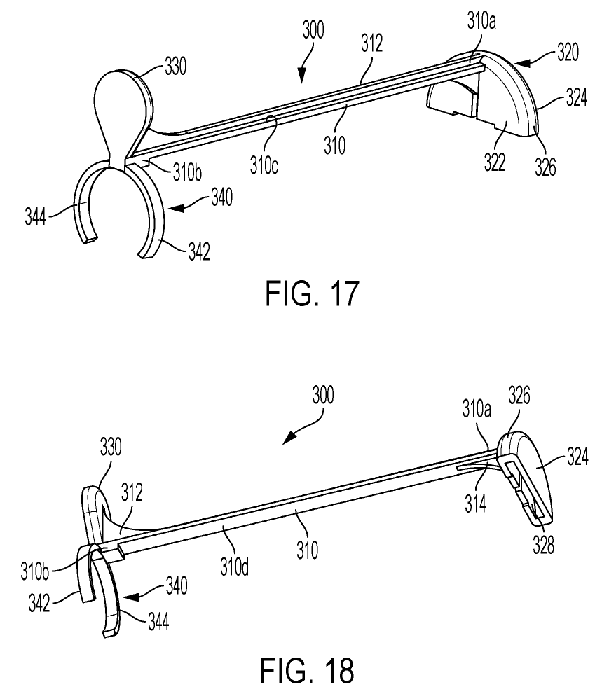

Referring to FIGS. 17-18, a device 300 for operating a syringe used in assisting insertion of a CVC into a patient, according to some embodiments of the present invention, is illustrated. The illustrated device 300 includes an elongate rod 310 having a proximal end 310a and a distal end 310b. A finger platform 330 extends outwardly from the rod distal end 310b, as illustrated and is configured to be engaged by a finger (e.g., the index finger) of a user holding a syringe to which the device 300 is attached. The finger platform 330 may be similar to the finger platform 160 described above, or may have a different configuration.

In the illustrated embodiment, the rod proximal end 310a is attached to a member 320 that is configured to engage a thumb press at the end of a plunger of a syringe (e.g., thumb press TP illustrated in FIGS. 13-16). The illustrated member 320 includes a pair of spaced-apart walls 322, 324 with a connecting peripheral wall 326 that define a slot 328 within which a portion of a thumb press can be positioned.

In the illustrated embodiment, the elongate rod 310 has a generally rectangular configuration with opposite first and second surfaces 310c, 310d. However, the rod 310 can have various shapes and configurations, and is not limited to the illustrated rectangular configuration. A reinforcing rib 312 extends outwardly from the first surface 310c and is connected at opposite ends to the finger platform 330 and to the member 320, as illustrated. Another reinforcing rib 314 extends outwardly from the second surface 310d and is connected to the member 320, as illustrated. These reinforcing ribs 312, 314 provide structural rigidity to the elongate rod 310.

The illustrated rod distal end 310b includes a C-shaped clip 340 that is configured to surround a portion of a syringe barrel when the device 300 is in use. The illustrated clip 340 has arcuate legs 342, 344 that are sized and configured to loosely straddle the barrel of a syringe and slide along the syringe barrel during use. The purpose of the clip 340 is to provide stability to the rod 310 during use or the device 300 in bidirectional movement of the plunger of the syringe.

The device 300 may be formed from various materials. In some embodiments, the device 300 may be formed from polymeric materials, such a polypropylene, polyethylene, etc. However, the device 300 may be formed from non-polymeric materials, such as stainless steel, etc.

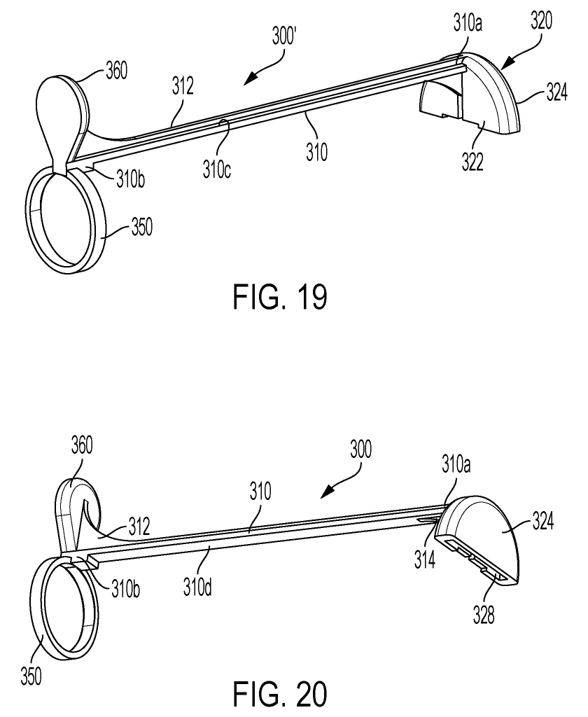

Referring to FIGS. 19-20, a device 300’ for operating a syringe used in assisting insertion of a CVC into a patient, according to some embodiments of the present invention, is illustrated. The illustrated device 300’ is similar to the device 300 illustrated in FIGS. 17-18 and includes an elongate rod 310 having a proximal end 310a and a distal end 310b. A finger platform 330 extends outwardly from the rod distal end 310b, as illustrated and is configured to be engaged by a finger of a user holding a syringe to which the device 300 is attached. The finger platform 330 is similar to the finger platform 160 described above, and is configured to be engaged by a finger (e.g., the index finger) of the user for operating the plunger of a syringe.

In the illustrated embodiment, the rod proximal end 310a is attached to a member 320 that is configured to engage a thumb press at the end of a plunger of a syringe (e.g., thumb press TP illustrated in FIGS. 13-16). The illustrated member 320 includes a pair of spaced-apart walls 322, 324 with a connecting peripheral wall 326 that define a slot 328 within which a portion of a thumb press can be positioned.

The illustrated rod distal end 310b includes a ring 350 that is sized and configured to loosely surround a syringe barrel when the device 300’ is in use. Similar to the clip 340 of the device 300 in FIGS. 17-18, the purpose of the ring 350 is to provide stability to the rod 310 during use of the device 300’ in bidirectional movement of the plunger of the syringe.

The device 300’ may be formed from various materials. In some embodiments, the device 300’ may be formed from polymeric materials, such a polypropylene, polyethylene, etc. However, the device 300’ may be formed from non-polymeric materials, such as stainless steel, etc.

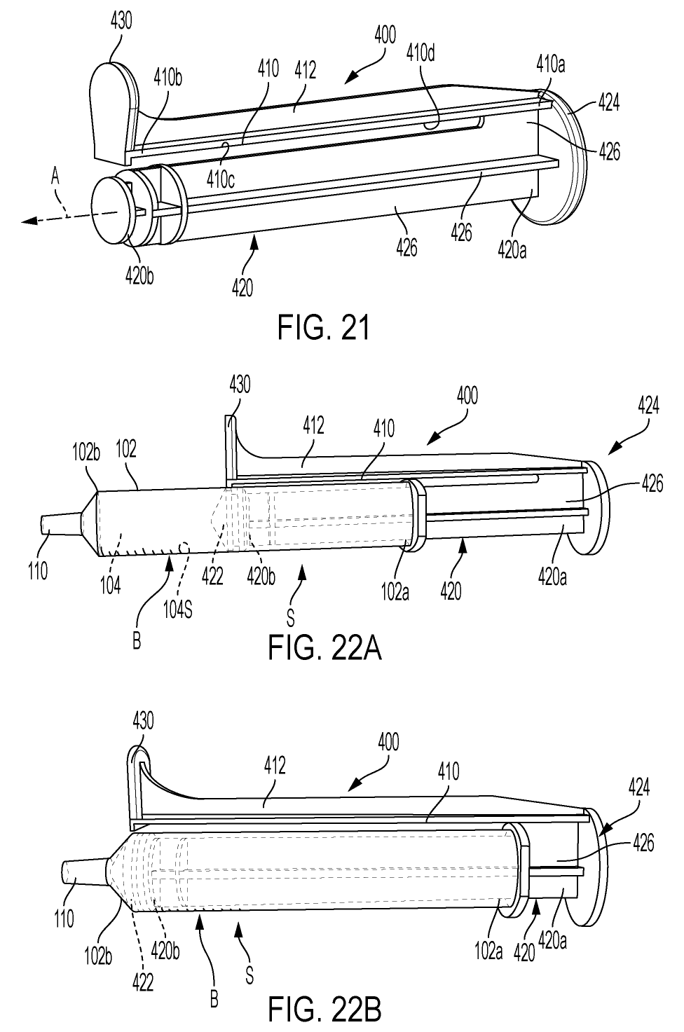

Referring to FIGS. 21 and 22A-22B, a device 400 for operating a syringe and assisting with insertion of a CVC into a patient, according to some embodiments of the present invention, is illustrated. The illustrated device 400 includes plunger 420 that is configured to be slidably mounted within the chamber 104 (FIGS. 22A-22B) of a syringe barrel B. The plunger 420 has an elongate configuration with a distal end 420b and an opposite proximal end 420a. A stopper 422 (FIGS. 22A-22B) is secured to the distal end 420b of the plunger 420 to form a fluid-tight seal with an inner surface 104s of the chamber 104. The stopper 122 may be made of a polymeric material, such as rubber for example, and is friction fit to the inner surface 104s of the chamber 104. As such, fluid contained within the chamber 104 can be expelled through the open distal end 102b of the barrel B (and through the tip 110) upon movement of the stopper 122 toward the distal end 102b of the barrel 102, and fluid may be drawn through the tip 110 and the open distal end 102b of the barrel 102 upon movement of the stopper 122 toward the proximal end 102a of the barrel 102.

The proximal end 420a of the plunger 420 includes a thumb press 424 that can be pressed by a user to move the plunger 420 toward the distal end 102b of the barrel 102, and can be gripped and pulled by a user to move the plunger 420 toward the proximal end 102a of the barrel 102. In the illustrated embodiment, the plunger 420 includes four elongate ribs 426 that extend radially from a longitudinal center axis A of the plunger 420. However, other embodiments of the plunger 420 may have different numbers of ribs. In addition, the plunger 420 may have various other shapes and configurations. Embodiments of the present invention are not limited to the illustrated shape of the plunger 420.

An elongate rod 410 includes a proximal end 410a that is attached to the thumb press 424 and to the plunger 420, as illustrated. A finger platform 430 is attached to the rod 410 near the rod distal end 410b and is configured to be engaged by a finger of a user holding the syringe 100. Bidirectional movement of the rod 410 via the finger platform 430 causes corresponding bidirectional movement of the plunger 420 within the barrel chamber 104. In other words, moving the finger platform 430 toward the proximal end 102a of the barrel 102 causes the plunger 420 to move toward the proximal end 102a of the barrel 102. Similarly, moving the finger platform 430 toward the distal end 102b of the barrel 102 causes the plunger 420 to move toward the distal end 102b of the barrel 102.

The elongate rod 410 has a generally rectangular configuration with opposite first and second surfaces 410c, 410d. A reinforcing rib 412 extends outwardly from the first surface 410c and is connected at opposite ends to the finger platform 430 and to the thumb press 424, as illustrated. The reinforcing rib 412 provides structural rigidity to the elongate rod 410. The plunger 420 is connected to the second surface 410d of the elongate rod 410, as illustrated.

Unlike the embodiment illustrated in FIGS. 3-8, the device 400 of FIG. 21 does not require the elongate rod 410 to be operably associated with a supporting guide or track on the barrel of a syringe. Instead, the elongated rod 410 of the device 400 is sufficiently rigid and strong to facilitate bidirectional movement of the plunger 420 within the syringe barrel 104 without requiring any supporting guides, tracks, clips or rings.

FIG. 22A illustrates the plunger 420 of the device 400 installed within a barrel 104 of a syringe S and with the stopper 422 positioned at a medial location within the syringe barrel 104. FIG. 22B illustrates the stopper 422 positioned at the distal end portion of the syringe barrel 104.

The device 400 may be formed from various materials. In some embodiments, the device 400 may be formed from one or more polymeric materials, such a polypropylene, polyethylene, etc. In other embodiments, the device 400 may be formed from non-polymeric materials, such as glass or stainless steel, etc.

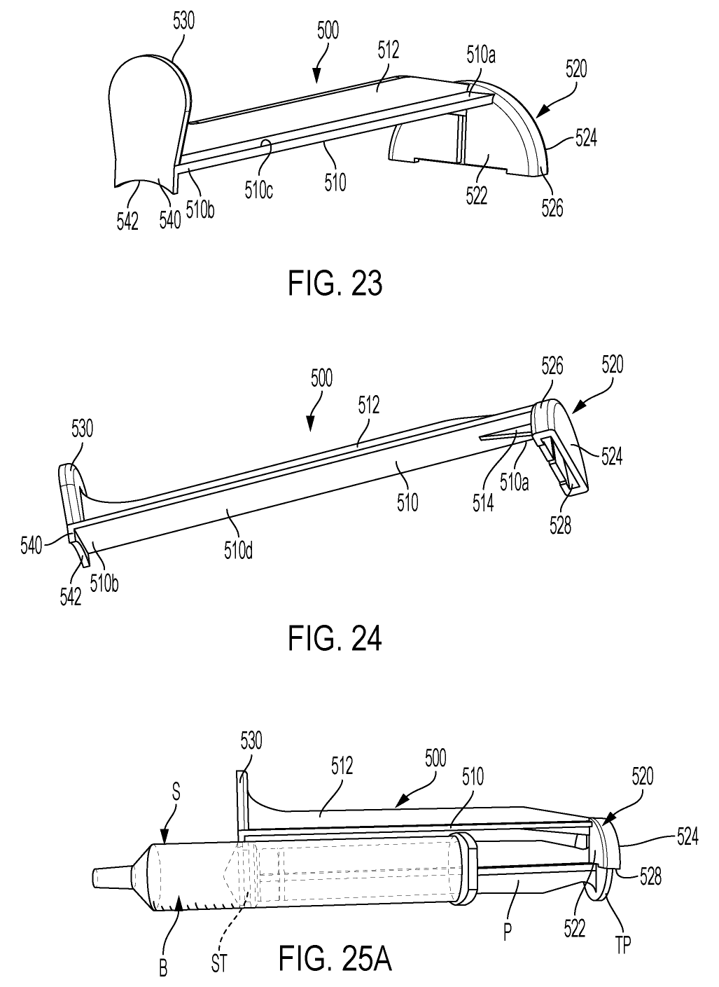

Referring to FIGS. 23-24, a device 500 for operating a syringe used in assisting insertion of a CVC into a patient, according to some embodiments of the present invention, is illustrated. The illustrated device 500 includes an elongate rod 510 having a proximal end 510a and a distal end 510b. A finger platform 530 extends outwardly from the rod distal end 510b, as illustrated and is configured to be engaged by a finger of a user holding a syringe to which the device 500 is attached. The finger platform 530 is similar to the finger platform 160 described above, and is configured to be engaged by a finger (e.g., the index finger) of the user for operating the plunger of a syringe.

In the illustrated embodiment, the rod proximal end 510a is attached to a member 520 that is configured to engage a thumb press at the end of a plunger of a syringe (e.g., thumb press TP illustrated in FIGS. 13-16). The illustrated member 520 includes a pair of spaced-apart walls 522, 524 with a connecting peripheral wall 526 that define a slot 528 within which a portion of a thumb press can be positioned.

In the illustrated embodiment, the elongate rod 510 has a generally rectangular configuration with opposite first and second surfaces 510c, 510d. However, the rod 510 can have various shapes and configurations, and is not limited to the illustrated rectangular configuration. A reinforcing rib 512 extends outwardly from the first surface 510c and is connected at opposite ends to the finger platform 530 and to the member 520, as illustrated. Another reinforcing rib 514 extends outwardly from the second surface 510d and is connected to the member 520, as illustrated. These reinforcing ribs 512, 514 provide structural rigidity to the elongate rod 510.

The illustrated rod distal end 510b includes a downwardly extending member 540 that is configured to slide along the outer surface of a syringe barrel when the device 500 is in use. The illustrated member 540 has arcuate surface 542 that is configured to engage the outer surface of a syringe barrel. In other embodiments, the member 540 need not have an arcuate surface.

In some embodiments, the device 500 may not have a member that engages the outer surface of a syringe barrel, i.e., the illustrated member 540 may be excluded). The elongated rod 510 may be sufficiently rigid as to not require any contact with the outer surface of a syringe barrel.

The device 500 may be formed from various materials. In some embodiments, the device 500 may be formed from polymeric materials, such a polypropylene, polyethylene, etc. However, the device 500 may be formed from non-polymeric materials, such as stainless steel, etc.

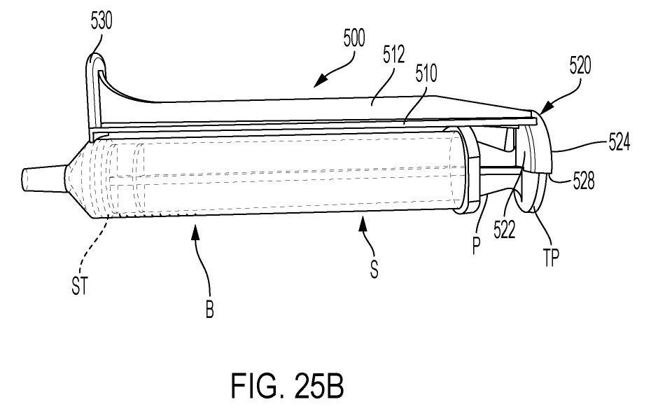

FIG. 25A illustrates the device 500 of FIGS. 23-24 attached to a syringe S and with the member 520 engaging the thumb press TP of the plunger P of the syringe S. The plunger P has been moved by the device 500 such that the stopper ST is positioned at a medial location within the syringe barrel B. FIG. 25B illustrates the stopper ST positioned at the distal end portion of the syringe barrel B.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity. The term “and/or” includes any and all combinations of one or more of the associated listed items.

Like numbers refer to like elements throughout. In the figures, certain components or features may be exaggerated for clarity, and broken lines may illustrate optional features or operations unless specified otherwise. In addition, the sequence of operations (or steps) is not limited to the order presented in the figures and/or claims unless specifically indicated otherwise. Features described with respect to one figure or embodiment can be associated with another embodiment or figure although not specifically described or shown as such.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected”, “coupled”, “responsive”, or variants thereof to another element, it can be directly connected, coupled or responsive to the other element or intervening elements may be present. In contrast, when an element is referred to as being “directly connected”, “directly coupled”, “directly responsive”, or variants thereof to another element, there are no intervening elements present. Other words used to describe the relationship between elements should be interpreted in a like fashion (i.e., “between” versus “directly between”, “adjacent” versus “directly adjacent”, etc.).

As used herein, the singular forms “a”, “an” and “the” are intended to include the plural forms as well, unless the context clearly indicates otherwise. As used herein, the terms “comprise”, “comprising”, “comprises”, “include”, “including”, “includes”, “have”, “has”, “having”, or variants thereof are open-ended, and include one or more stated features, integers, elements, steps, components or functions but does not preclude the presence or addition of one or more other features, integers, elements, steps, components, functions or groups thereof. Furthermore, as used herein, the common abbreviation “e.g.,” which derives from the Latin phrase “exempli gratia,” may be used to introduce or specify a general example or examples of a previously mentioned item, and is not intended to be limiting of such item. The common abbreviation “i.e.,” which derives from the Latin phrase “id est,” may be used to specify a particular item from a more general recitation.

It will be understood that although the terms first, second, third, etc., may be used herein to describe various elements/operations, these elements/operations should not be limited by these terms. These terms are only used to distinguish one element/operation from another element/operation. Thus, a first element/operation in some embodiments could be termed a second element/operation in other embodiments without departing from the teachings of the present invention. The same reference numerals or the same reference designators denote the same or similar elements throughout the specification.

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terms “about” and “approximately”, as used herein with respect to a value or number, is meant to encompass variations of ± 10%, ± 5%, ± 1%, ± 0.5%, or even ± 0.1% of the specified value as well as the specified value. For example, “about X” where X is the measurable value, is meant to include X as well as variations of ± 10%, ± 5%, ± 1%, ± 0.5%, or even ± 0.1% of X. A range provided herein for a measurable value may include any other range and/or individual value therein.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the teachings and advantages of this invention. Accordingly, all such modifications are intended to be included within the scope of this invention as defined in the claims. The invention is defined by the following claims, with equivalents of the claims to be included therein.

What is claimed is:

1. A syringe, comprising:
a barrel defining a longitudinally extending chamber and comprising an open proximal end, and an open distal end;
a plunger slidably mounted within the chamber; and
an elongate rod movably secured to an outer portion of the barrel, wherein the rod comprises a proximal end attached to the plunger, a distal end, and a finger platform adjacent the rod distal end that is configured to be engaged by a finger of a user holding the syringe, and wherein bidirectional movement of the rod via the finger platform causes corresponding bidirectional movement of the plunger within the barrel.

2. The syringe of claim 1, further comprising a stopper secured to a distal end of the plunger to form a fluid-tight seal with an inner surface of the chamber.

3. The syringe of claim 1, wherein the barrel comprises a longitudinally extending guide integral with an outer surface thereof, and wherein the rod is slidably retained within the guide.

4. The syringe of claim 1, wherein the finger platform has an ergonomic configuration that facilitates engagement by the finger of the user.

5. The syringe of claim 1, wherein the finger platform comprises a rounded periphery, a flat distal surface, and a curved proximal surface.

6. The syringe of claim 1, wherein a proximal end of the plunger comprises a thumb press, and wherein the rod proximal end is attached to the thumb press.

7. The syringe of claim 1, wherein the barrel distal end comprises a wall with a tip extending distally therefrom having a passageway therethrough in fluid communication with the chamber.

8. The syringe of claim 1, wherein fluid contained within the chamber is expelled through the open distal end of the barrel upon movement of the stopper toward the distal end of the barrel, and wherein fluid may be drawn through the open distal end of the barrel and into the chamber upon movement of the stopper toward the proximal end of the barrel.

9. A device for operating a syringe, the device comprising:
a frame configured to be attached to a barrel of the syringe; and
an elongate rod movably secured to the frame, wherein the rod comprises a proximal end, a distal end, and a finger platform configured to be engaged by a finger of a user holding the syringe, wherein the rod proximal end is configured to engage a portion of a plunger extending outward from the barrel proximal end, and wherein bidirectional movement of the rod via the finger platform causes corresponding bidirectional movement of the plunger within the barrel of the syringe.

10. The device of claim 9, wherein the frame comprises a first clip configured to be removably secured to a distal end portion of the syringe barrel and a second clip configured to be removably secured to a proximal end portion of the syringe barrel.

11. The device of claim 9, wherein the proximal end of the syringe barrel comprises a peripheral flange, and wherein the frame comprises an adapter configured to surround a portion of the barrel and engage the peripheral flange.

12. The device of claim 11, wherein the adapter comprises a pair of spaced-apart clips configured to receive the portion of the peripheral flange therebetween.

13. The device of claim 9, wherein the plunger comprises a thumb press, and wherein the rod proximal end is configured to engage the thumb press.

14. The device of claim 13, wherein the rod proximal end comprises a member with a slot, and wherein a portion of the thumb press is received within the slot.

15. The device of claim 9, wherein the frame comprises a longitudinally extending guide on an outer surface thereof, and wherein the rod is slidably retained within the guide.

16. The device of claim 9, wherein the finger platform has an ergonomic configuration that facilitates engagement by the finger of the user.

17. A device for operating a syringe, the device comprising:
an elongate rod comprising a proximal end and an opposite distal end, wherein the proximal end of the rod is removably secured to a thumb press of a plunger movably secured within a barrel of the syringe, and wherein the distal end of the rod comprises a finger platform that is configured to be engaged by a finger of a user holding the syringe, wherein bidirectional movement of the rod via the finger platform causes corresponding bidirectional movement of the plunger within the barrel of the syringe.

18. The device of claim 17, wherein the rod proximal end comprises a member with a slot, and wherein a portion of the thumb press is received within the slot.

19. The device of claim 17, wherein the rod distal end comprises a member configured to slide along an outer surface of the barrel of the syringe during movement of the rod.

20. The device of claim 17, wherein the rod distal end comprises an arcuate member that at least partially surrounds the barrel of the syringe and is configured to slide along an outer surface of the barrel of the syringe during movement of the rod.
